(19) United States
(12) Patent Application Publication
(10) Pub. No.: US 2016/0008164 A1
(43) Pub. Date: Jan. 14, 2016

(54) HANDHELD BODY TEMPERATURE REGULATOR

(71) Applicants: Kimbal W Anderson, Pendleton, OR (US); Kimberley A Anderson, Pendleton, OR (US)

(72) Inventors: Kimbal W Anderson, Pendleton, OR (US); Kimberley A Anderson, Pendleton, OR (US)

(21) Appl. No.: 14/798,882

(22) Filed: Jul. 14, 2015

Related U.S. Application Data
(60) Provisional application No. 62/024,190, filed on Jul. 14, 2014.
(60) Provisional application No. 62/131,535, filed on Mar. 11, 2015.
(60) Provisional application No. 62/187,092, filed on Jun. 30, 2015.

(51) Int. Cl. A61F 7/00

(52) U.S. Cl. CPC A61F 7/0053; A61F 2007/0036; A61F 2007/0054; A61F 2007/0087

(57) ABSTRACT

A portable heat exchanging apparatus for regulating body temperature. At least one coil of thermally conductive tubing is formed to define a plurality of consecutive, concentric windings. A connection tubing connects the at least one coil to an external fluid supply. In some embodiments, a protective covering tightly encloses the at least one coil, providing a smooth surface for user comfort, easy sanitization, and reusability.

### CROSS-REFERENCE TO RELATED APPLICATIONS

This application claims priority to U.S. Provisional Patent Application Ser. No. 62/024,190 filed Jul. 14, 2014 and entitled HANDHELD BODY TEMPERATURE REGULATOR (HBTR), U.S. Provisional Patent Application Ser. No. 62/131,535 filed Mar. 11, 2015 and entitled HANDHELD BODY TEMPERATURE REGULATOR II (HBTR II), and U.S. Provisional Patent Application Ser. No. 62/187,092 filed Jun. 30, 2015 and entitled HANDHELD BODY TEMPERATURE REGULATOR (HBTR) the disclosures of which are incorporated herein by reference.

### SUMMARY OF THE INVENTION

The present invention is directed toward a portable heat exchanging apparatus and method for safely and efficiently regulating the body temperature of a user, primarily in a medical pre/postoperative and surgical setting. Specifically, the apparatus and method are capable of warming or cooling the body temperature of a user by forcing or circulating warm or cold fluid from an external fluid supply to flow through at least one thermally conductive coil. Heat is transferred between the apparatus and a user, through contact between at least one thermally conductive coil and an exposed body part of a user such as a palm, foot, nose, or ear. The claimed invention may be used in homes, hospitals, paramedic vehicles, medical tents, taxi cabs, airplanes, boats, etc., or in the field as part of any medical aid kit. Additionally, because the claimed apparatus is portable, it may be easily transported via bicycles, motor vehicles, aircrafts, watercrafts, in backpacks, etc., and used in remote locations. As such, the claimed invention may be taken on location when a user may require body temperature regulation, for example, during hiking, backpacking, or bicycle touring trips.

Common uses of the claimed invention include prevention of hypothermia under anesthesia and treatment of heat stress events, such as heatstroke or heat exhaustion, while avoiding the problems associated with apparatuses and methods that are currently used for regulating body temperature. Hypothermia under anesthesia is a significant problem for patients during surgery and may lead to cardiac events, increased infections, increased blood loss, increased blood pressure, impaired metabolism of drugs, patient discomfort, increased hospital costs, and even death. Common apparatuses currently used for preventing hypothermia under anesthesia include disposable forced warm air coverings and disposable circulating water mattresses that are bulky, cumbersome to move, expensive, single-use, inefficient, and inconveniently require that a user remain in a recumbent position. By contrast, the claimed is lighter, portable, inexpensive, reusable, efficient, and convenient to use.

Common methods currently used for preventing hypothermia under anesthesia also include warming the body temperature of a user by warning the operating room, which may be uncomfortable for hospital staff. By contrast, the claimed invention allows for the isolated warming of the body temperature of a user without having to warm the temperature of the operating room thereby causing discomfort to hospital staff. Additionally, common methods currently used to treat heat stress events include applying ice packs to the head, underarms, and groin of a patient, which may be uncomfortable or even painful for the patient, and loses effectiveness over time due to vasoconstriction that decreases the blood flow of a user. By contrast, the claimed invention may comfortably and consistently cool the body temperature of a user, without causing vasoconstriction or losing effectiveness over time.

The claimed apparatus may also have uses additional to body temperature regulation. One such use is preparing a patient for stressful events such as blood draw or intravenous start procedures. Specifically, the claimed apparatus may be used to quickly dilate the blood vessels of a user to their maximum size, thereby increasing the success rate of the procedures. Additionally, the claimed apparatus may offer the user stress relieving effects during these procedures, due to the shape of the coil, which may be ergonomic and thus easy to hold and squeeze. The claimed invention may also provide the user with stress relieving effects before, during, or after surgery. Although the claimed invention is useful in hospital settings, it may be used in remote locations without the need for specialized healthcare providers.

### BACKGROUND OF THE DISCLOSURE

Examples of related systems are disclosed in U.S. Pat. Nos. 8,758,419, 8,603,150, 7,160,315, 5,336,249, and 4,844,072. The disclosures of these and all other publications referenced herein are incorporated by reference in their entirety for all purposes.

Advantages of the present disclosure will be more readily understood after considering the drawings and the Detailed Description.

### BRIEF DESCRIPTION OF THE DRAWINGS

FIG. 1A is a partially transparent perspective view showing a pair of coils of thermally conductive tubing, each enclosed in a protective covering, according to one embodiment of the invention.

FIG. 1B is a side view showing a pair of coils of thermally conductive tubing, without any protective covering, connected in series, and completing a fluid circuit.

FIG. 1C-1D are perspective views showing one of the coils of FIG. 1B.

FIG. 1E is a top view showing a distal end of the coil of FIGS. 1C-1D.

FIG. 1F is a bottom view showing a base of the coil(s) of FIGS. 1C-1D, with a cutaway view of the thermally conductive tubing at the base of the coil.

FIG. 2A is a partially transparent perspective view showing a pair of frustoconical coils of thermally conductive tubing, each enclosed in a protective covering, according to one embodiment of the invention.

FIG. 2B is a side view showing one frustoconical coil of FIG. 2A, with the protective covering cut away to expose one half of the coil.

FIG. 2C is a top view showing a distal end of the frustoconical coil of FIG. 2B, with the protective covering cut away to expose the top of the coil.

FIG. 2D is a bottom view showing a base of the frustoconical coil of FIG. 2B with a cutaway view of the thermally conductive tubing at the base of the coil.

FIG. 3 is a perspective side view of a pair of ergonomic, frustoconical coils of thermally conductive tubing, without any protective covering, according to one embodiment of the invention.

FIG. 4A is a perspective side view of a pair of ergonomic, frustoconical coils of thermally conductive tubing, wherein each coil is tightly enclosed in a protective covering, according to one embodiment of the invention.

FIG. 4B is a side view showing one ergonomic, frustoconical coil of FIG. 4A, with the protective covering cut away to expose one half of the coil.

FIG. 4C is a top view showing the ergonomic, frustoconical coil of FIG. 4B, with the protective covering cut away to expose the top of the coil.

FIG. 4D is a bottom view showing a base of the frustoconical coil of FIG. 4B with a cutaway view of the thermally conductive tubing at the base of the coil.

FIG. 5 is a partially transparent perspective view showing a pair of frustoconical coils of thermally conductive tubing, each enclosed in a protective covering, according to one embodiment of the invention.

FIG. 6 is a perspective side view of a pair of ergonomic, frustoconical coils of thermally conductive tubing, wherein each coil is tightly enclosed in a protective covering, according to one embodiment of the invention.

### DETAILED DESCRIPTION OF THE EMBODIMENTS

Embodiments of the invention include an apparatus and a method for regulating the body temperature and dilating blood vessels of a user by forcing or circulating warm or cold fluid from an external fluid supply to flow through at least one coil of thermally conductive tubing that may be in contact with an exposed body part of a user.

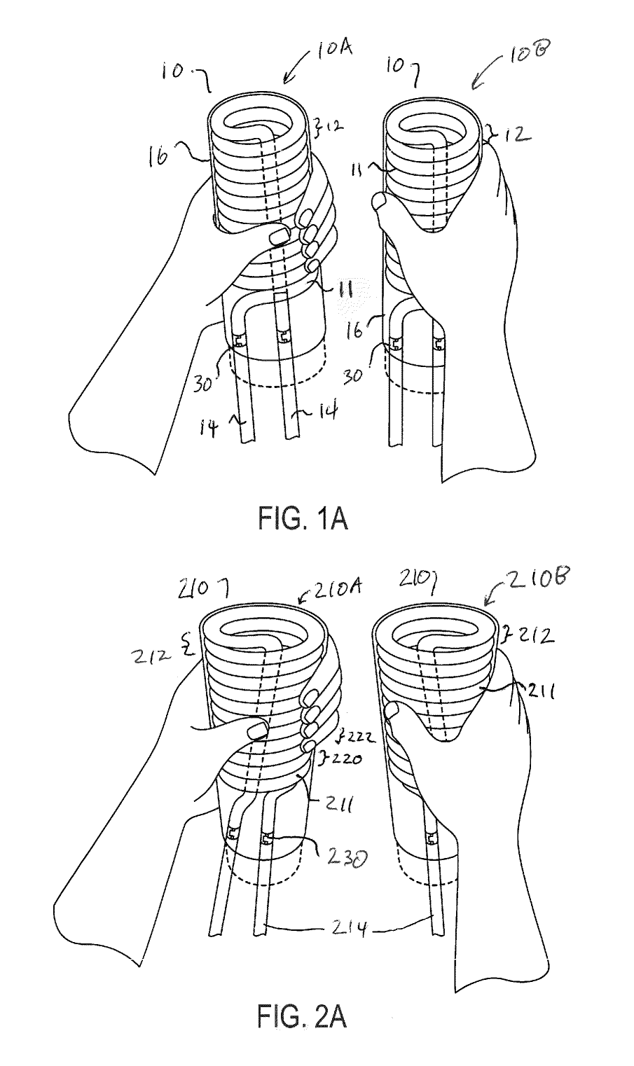

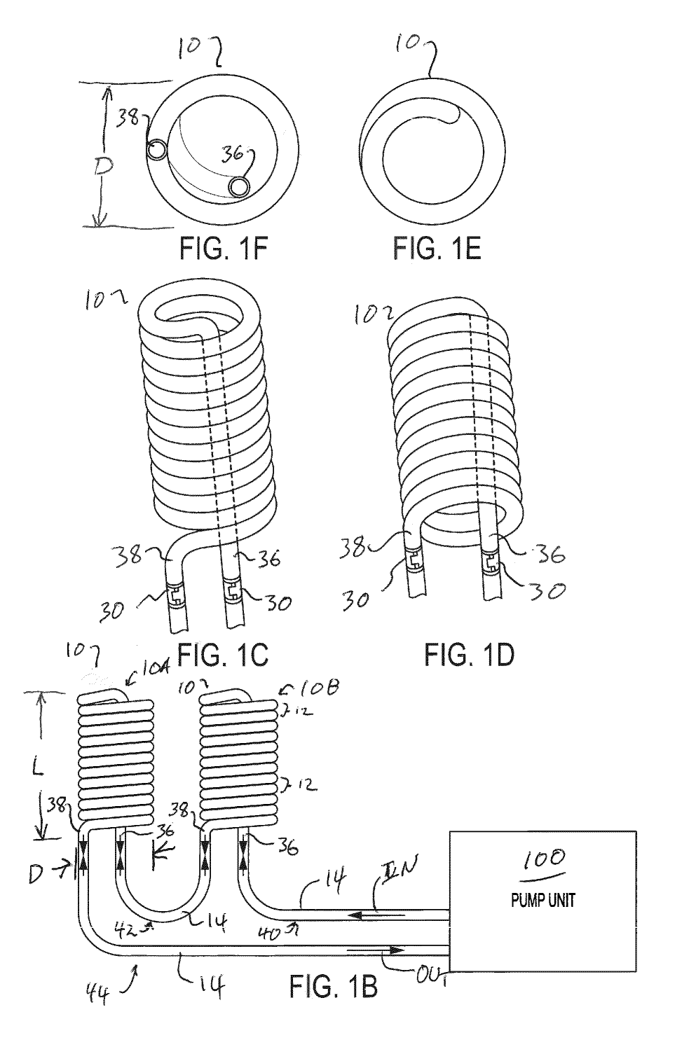

As shown in FIG. 1A, an embodiment may include at least one coil 10 of thermally conductive tubing 11, defining a plurality of consecutive, concentric windings 12 of thermally conductive tubing 11. Some embodiments may be configured for handheld use; an example of possible hand positioning is shown in FIG. 1A. Accordingly, some embodiments may include a first coil 10A and a second coil 10B connected in series via connection tubing 14 and secured at points of connection by a sealing element 30. A benefit of having a pair of coils may be that in embodiments configured for handheld use, a user may hold a coil 10 in each hand, which may allow for more effective body temperature regulation than if only a single coil were used. For example, as shown in FIG. 1A, coil 10A may be held in a left hand, and coil 10B may be held in a right hand. However, embodiments may include any other appropriate number of coils, such as one coil, or multiple coils. Additionally, other embodiments may be configured for non-handheld use. Specifically, the coil(s) 10 may be in contact with any exposed body part(s) of a user, such as a foot, ear, etc. Heat may be exchanged between the coil(s) 10 and any exposed body part(s) of a user. Temperature of the fluid may be varied to achieve the desired warming or cooling of the body temperature of a user.

An embodiment may be configured to be portable, thus the coil(s) 10 may be of any size suitable for portability. Specifically, the at least one coil 10 may have a length L of approximately 6-inches or any other suitable length, and include consecutive, concentric windings 12 having diameter(s) D of approximately 1.75-inches or any other suitable diameter(s). The length(s) of the coil(s) 10 and diameter(s) of the consecutive, concentric windings 12 may vary in embodiments intended for different users, such as children and/or adults. For example, embodiments intended for use by children may have less consecutive, concentric windings 12 and thus be shorter in length than an embodiment intended for use by adults. Additionally, embodiments intended for use by children may have consecutive, concentric windings of smaller diameter(s) and thus be more easily gripped in the hand(s) of a child than embodiments intended for use by adults.

The thermally conductive tubing 11 may be any thermally conductive material or mixture of materials. Additionally, the thermally conductive tubing 11 may be malleable. Malleability may provide easier formation of the coil(s) 10 by winding the thermally conductive tubing 11. In an embodiment, the thermally conductive tubing 11 may be copper, which is malleable and suitable for winding. In another embodiment, the thermally conductive tubing 11 may be copper with nickel plating, which is malleable, suitable for winding, and may help eliminate the smell of copper. Further, nickel plating may allow for some flexing when squeezed in a hand of a user, which may facilitate use of the coil(s) 10 as a stress relief device. For stress relief, a user may tightly grip the coil(s) 10 in the hand(s) of a user. The thermally conductive tubing 11 may have an approximate outer diameter of ¼-inch or any other diameter suitable for winding.

The thermally conductive tubing 11 and connection tubing 14 are hollow to allow the flow of fluid therewithin. Although directional arrows IN and OUT have been drawn in FIG. 1B to indicate a direction in which fluid may flow, fluid may alternatively flow through the coil(s) 10 and connection tubing 14 in an opposite direction. In one embodiment, warm or cold fluid from an external fluid supply 100 may be forced through the connection tubing 14 and the coil(s) 10, enter through an input end 36 of the thermally conductive tubing 11, and exit through an output end 38 of the thermally conductive tubing 11. The exiting fluid may be disposed of in any suitable manner, such as by draining, storing, recirculating, etc.

In some embodiments, as shown in FIG. 1B, the connection tubing 14 may complete a fluid circuit with the coil(s) 10A and 10B and an external fluid supply 100. In such embodiments, the external fluid supply 100 may be a fluid circulating pump, such that fluid may be circulated through the connection tubing 14 and coil(s) 10. Although the embodiment shown in FIG. 1B includes a fluid circuit, alternative embodiments may not include a fluid circuit. In these alternative embodiments, the external fluid supply 100 may be a faucet or any other suitable source of fluid. The flow or circulation of warm water may warm the body temperature of a user. Alternatively, the flow or circulation of cold water may cool the body temperature of a user.

The connection tubing 14 may be a flexible material or mixture of materials such as but not limited to plastic, and be secured at points of connection by a sealing element (not pictured in FIG. 1B). The connection tubing 14 may include a plurality of segments. For example, as shown in FIG. 1B, the connection tubing 14 may include three segments, or any other appropriate number of segments. For example, a first connection tubing segment 40 may connect the coil(s) 10 with an external fluid source 100 such as a fluid circulation pump or any other suitable source of fluid, and allow for fluid input. A second connection tubing segment 42 may connect two coil(s) 10. Similar to the first connection tubing segment 40, a third connection tubing segment 44 may complete a fluid circuit with a fluid circulation pump, and/or allow for fluid output. Fluid may be output to any suitable location such as a fluid reservoir or drain, or may be recirculated through the apparatus via a fluid circulating pump. Although arrows have been drawn to indicate one direction of fluid flow and/or circulation, fluid may flow and/or circulate in the opposite direction.

Additionally, as shown in FIG. 1A, the coil(s) 10 may be tightly enclosed in a protective covering 16 that may provide a smooth surface for user comfort, easy sanitation, and reusability, while still allowing efficient heat transfer between the coil and an exposed body part of a user. The protective covering 16 may tightly enclose the coil(s) and be thin material such as polyolefin shrink tubing with a 2:1 or 3:1 shrink ratio. Other tubing materials such as foam, rubber, silicone, or any mixture thereof may also be used. Although a thicker protective covering may provide a cushier surface and thus be more comfortable for hand gripping, it may also add insulation and thereby decrease efficiency of heat transfer. In some embodiments of the invention, the protective covering 16 may additionally enclose at least a portion of the connection tubing 14, as indicated by the dashed line in FIG. 1A.

In other embodiments, the protective covering 16 may be removable and/or disposable and a material such as but not limited to paper, fabric, plastic, or any mixture thereof. The protective covering 16 may further be configured in any suitable manner. For example, the protective covering 16 may provide the coil(s) 10 with smooth, contiguous outer and/or inner surface(s) by tightly enclosing part of or the entire outer and/or inner surface(s) of the coil(s) 10. In other embodiments, the protective covering 16 may provide the coil(s) 10 with ribbed outer and inner surfaces by tightly enclosing the thermally conductive tubing 11 rather than the entire coil(s) 10. Alternatively, the protective covering 16 may be configured to provide the coil(s) 10 with a partial surface that is smooth and contiguous, and partial surface that is ribbed.

The coil(s) 10 may include a length of thermally conductive tubing 11 that bends from the final concentric winding 24 and extends along the coil(s) 10 toward a base 32 of the coil(s) 10, in a direction substantially perpendicular to an axis defined by the plurality of consecutive, concentric windings 12. The length of thermally conductive tubing 11 may be positioned inside the plurality of consecutive, concentric windings 12, as shown in FIGS. 1A-1F. Although FIGS. 1E-1F show the length of thermally conductive tubing 11 to be positioned flush with the plurality of consecutive, concentric windings 12, the length of thermally conductive tubing 11 may be positioned any suitable distance from the plurality of consecutive, concentric windings 12. Additionally, the length of thermally conductive tubing 11 may protrude beyond the first concentric winding 20.

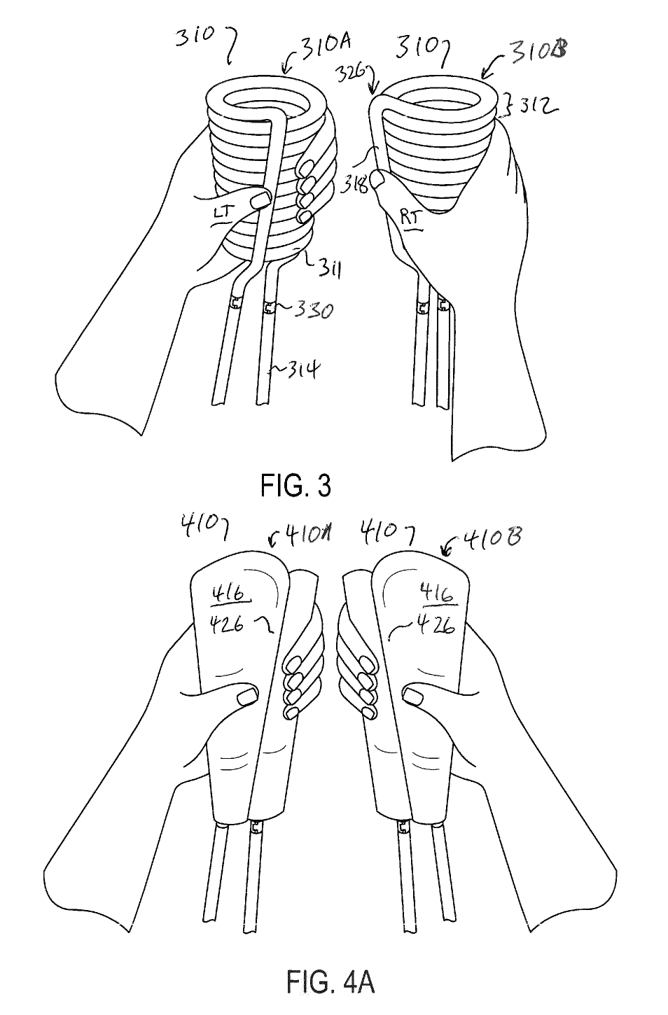

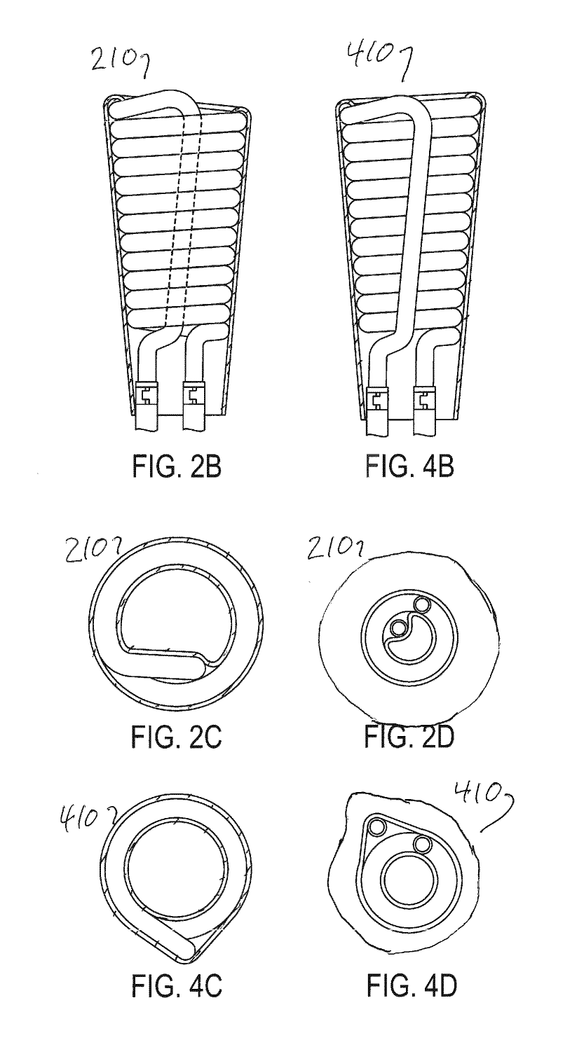

Further, although the coil(s) 10 shown in FIG. 1A are cylindrical, the coil(s) 10 may be of any suitable shape. For instance, as shown in FIG. 2A and discussed further below, the coil(s) 210 may be frustoconical, in which the plurality of consecutive, concentric windings 212 of thermally conductive tubing 211 defines incrementally increasing diameters. For simplicity, corresponding elements in the embodiment of FIGS. 2A-2D have been labeled using similar reference characters, but with an added leading “2.”

For example, as shown in the drawings, a first concentric winding 220 may have a first diameter that is smaller than an adjacent concentric winding 222 having a second diameter. Other embodiments of the invention may include differently shaped coil(s), defining consecutive, concentric windings of different shapes (such as circular, oval, or oblong) and diameters. The consecutive, concentric windings 212 may be contiguous, that is, the consecutive, concentric windings 212 may have substantial spaces therebetween.

In other embodiments, the length of thermally conductive tubing 11 may be positioned outside and flush with the plurality of consecutive, concentric windings 12, creating a lengthwise outer ridge along the coil(s) 10, similar to what is shown in FIG. 3. The lengthwise outer ridge may provide an indicator for right or left thumb placement. Alternatively, the length of thermally conductive tubing 11 may be positioned outside and not flush with the plurality of consecutive, concentric windings 12. An embodiment of the invention with more than one coil 10 may include any combination of coils with lengths of thermally conductive tubing 11 inside or outside of the plurality of consecutive, concentric windings 12. For example, a pair of coils 10 may include a first coil with a length of thermally conductive tubing 11 inside the plurality of consecutive, concentric windings 12, and a second coil with a length of thermally conductive tubing 11 outside the plurality of consecutive, concentric windings 12.

FIG. 1C shows a perspective view of a distal end 34 and side of the coil(s) 10 according to one embodiment. The distal end 34 may include a final concentric winding 24 of thermally conductive tubing 11. Although FIG. 1C shows the length of thermally conductive tubing 11 positioned inside and flush with the consecutive, concentric windings 12, other embodiments may be configured such that the length of thermally conductive tubing 11 is positioned inside and not flush with the consecutive, concentric windings 12, outside and flush with the consecutive, concentric windings 12, or outside and not flush with the consecutive, concentric windings 12.

FIG. 1D shows a perspective view of a base 32 and side of the coil(s) 10 according to one embodiment. The base 32 of the coil 10 may include an input end 36 of the thermally conductive tubing 11, an output end 38 of the thermally conductive tubing 11, and a first concentric winding 20 of the thermally conductive tubing 11. Accordingly, the base 32 may also be referred to as a feed-and-return end. Although arrows have been drawn to indicate one possible direction of fluid flow and/or circulation, fluid may also flow and/or circulate in the opposite direction. Accordingly, the input end 36 may be used for fluid output, and the output end 38 may be used for fluid input.

FIG. 1E shows a top view of a distal end 34 of the coil(s) 10 according to one embodiment. The distal end 34 may include a final concentric winding 24 of thermally conductive tubing 11. Although FIG. 1E shows the length of thermally conductive tubing 11 positioned inside and flush with the consecutive, concentric windings 12, other embodiments may be configured such that the length of thermally conductive tubing 11 positioned inside and not flush with the consecutive, concentric windings 12; outside and flush with the consecutive, concentric windings 12; or outside and not flush with the consecutive, concentric windings 12.

FIG. 1F shows a bottom view of a base 32 of the coil(s) 10 according to one embodiment. The thermally conductive tubing 11 has been cut away and arrows have been drawn to show a direction in which fluid may flow and/or circulate therewithin. The base 32 of the coil 10 may include an input end 36 of the thermally conductive tubing 11, an output end 38 of the thermally conductive tubing 11, and a first winding 24 of the thermally conductive tubing 11. Accordingly, the base 32 may also be referred to as a feed-and-return end. Although arrows have been drawn to indicate one possible direction of fluid flow and/or circulation, fluid may also flow and/or circulate in the opposite direction. Accordingly, the input end 36 may be used for fluid output, and the output end 38 may be used for fluid input.

As shown in FIG. 2A, another embodiment may include at least one frustoconical coil 210 of thermally conductive tubing 211, defining a plurality of consecutive, concentric windings 212 of thermally conductive tubing 211. Some embodiments may be configured for handheld use; examples of possible hand positioning are shown in FIGS. 2A and 2B. Accordingly, some embodiments may include a first frustoconical coil 210A and a second frustoconical coil 210B connected in series via connection tubing 214 and secured at points of connection by a sealing element 230. Because the frustoconical coil(s) 210 more closely resemble the shape naturally taken by a relaxed hand, the frustoconical coil(s) 210 may provide more user comfort than other embodiments, for example the coil(s) 10 shown in FIG. 1A, when held in the hand(s) of a user. However, other embodiments may be configured for non-handheld use. Specifically, the frustoconical coil(s) 210 may be in contact with any exposed body part(s) of a user, such as a foot, ear, etc. Heat may be exchanged between the frustoconical coil(s) 210 and any exposed body part(s) of a user. Temperature of the fluid may be varied to achieve the desired warming or cooling of the body temperature of a user.

An embodiment may be configured to be portable, thus the frustoconical coil(s) 210 may be of any size suitable for portability. Specifically, the at least one frustoconical coil 210 may have a length of approximately 5-inches or any other suitable length. In some embodiments, as shown in FIGS. 2A and 2B, the distal end(s) 234 of the frustoconical coil(s) 210 may be larger than the base(s) 232 of the frustoconical coil(s) 210. In other words, the plurality of consecutive, concentric windings 212 of thermally conductive tubing 211 may define incrementally increasing diameters. Specifically, the first concentric winding 220 may have a first diameter of approximately 1.5-inches at the base(s) 232 of the frustoconical coil(s) 210, and the final concentric winding 224 may have a final diameter of approximately 2.25-inches at the distal end(s) 234 of the frustoconical coil(s) 210, or any other suitable first and final diameters.

Further, although FIGS. 2A and 2B show hand placements in which the narrower end(s) of the frustoconical coil(s) 210 are held nearer the wrist(s) of a user, the frustoconical coil(s) 210 may be held in other suitable hand position(s). Alternatively, the frustoconical coil(s) 210 may be placed on any exposed body part(s) of a user such as a foot, ear, etc.

In some embodiments, the consecutive, concentric windings 212 may be contiguous, that is, the consecutive, concentric windings 212 may not have substantial spaces therebetween. In other words, a first concentric winding 220 of thermally conductive tubing 211 may be positioned flush with an adjacent concentric winding 222 of thermally conductive tubing 211. The frustoconical coil(s) may be of any suitable size and include any suitable number of consecutive, concentric windings of thermally conductive tubing 211. For example, an embodiment may include at least five consecutive, concentric windings of thermally conductive tubing 211. The number of consecutive, concentric windings may vary in embodiments intended for different users, such as, for example, children and/or adults.

The thermally conductive tubing 211 may be any thermally conductive material or mixture of materials, as discussed above with respect to thermally conductive tubing 11. Similarly, the connection tubing 214 may be a flexible material or mixture of materials, as discussed above.

Additionally, as shown in FIGS. 2A-2D, the frustoconical coil(s) 210 may be tightly enclosed in a protective covering 216 that may provide a smooth surface for user comfort, easy sanitation, and reusability, while still allowing efficient heat transfer between the frustoconical coil(s) and an exposed body part of a user.

The frustoconical coil(s) 210 may include a length of thermally conductive tubing 218 that bends from the final concentric winding 224 and extends along the frustoconical coil(s) 210 toward a base 232 of the frustoconical coil(s) 210, in a direction substantially perpendicular to an axis defined by the plurality of consecutive, concentric windings 212. The length of thermally conductive tubing 218 may be positioned inside the plurality of consecutive, concentric windings 212, as indicated by the dashed lines in FIGS. 2A-2B.

FIG. 2C shows a top view of a distal end 234 of the frustoconical coil(s) 210 according to one embodiment. The distal end 234 may include a final concentric winding 124 of thermally conductive tubing 211. Additionally, FIG. 2C shows the length of thermally conductive tubing 218 positioned inside and flush with the consecutive, concentric windings 212. In other embodiments, a length of thermally conductive tubing may be positioned inside and not flush with the consecutive, concentric windings; outside and flush with the consecutive, concentric windings; or outside and not flush with the consecutive, concentric windings.

FIG. 2D shows a bottom view of a base of the frustoconical coil(s) 210 according to one embodiment. The base 232 of the frustoconical coil(s) 210 may include a first concentric winding 220 of thermally conductive tubing, a beginning of thermally conductive tubing 236 for fluid input, and an end of thermally conductive tubing 238 for fluid output. Accordingly, the base 232 may also be referred to as a feed-and-return end. As mentioned above, fluid may alternatively flow and/or circulate in the opposite direction. Accordingly, in alternative embodiments, the input end 236 may be used for fluid output, and the output end 238 may be used for fluid input.

As shown in FIG. 3, another embodiment may include at least one ergonomic, frustoconical coil 310 of thermally conductive tubing 311, defining a plurality of consecutive, concentric windings 312 of thermally conductive tubing 311. Some embodiments may be configured for handheld use, as shown in FIG. 3. Accordingly, some embodiments may include a first ergonomic, frustoconical coil 310A configured to be held in one hand of a user, and a second ergonomic, frustoconical coil 310B configured to be held in another hand of a user, connected in series via connection tubing 314 and secured at points of connection by a sealing element 330. Although the ergonomic, frustoconical coil(s) 310 are configured for handheld use, the ergonomic, frustoconical coil(s) 310 may be placed on any exposed body part(s) of a user, such as a foot, ear, etc. Heat may be exchanged between the ergonomic, frustoconical coil(s) 310 and any exposed body part(s) of a user. Temperature of the fluid may be varied to achieve the desired warming or cooling of the body temperature of a user.

The embodiment of FIG. 3 is configured to be portable, thus the ergonomic, frustoconical coil(s) 310 are of a size suitable for portability. Specifically, the at least one ergonomic, frustoconical coil 310 may have a length of approximately 6-inches and a first diameter of approximately 1-inch at the base, and a final diameter of approximately 1.75-inches at the distal end, or any other suitable length and diameters. Similar to the coil(s) 10 of FIG. 1A and the frustoconical coil(s) 210 of FIG. 2A, the size(s) of the ergonomic, frustoconical coil(s) 310 may vary in embodiments intended for different users, such as children and/or adults. Further, the consecutive, concentric windings 312 may be of any ergonomic shape, preferably oval or oblong, for a potentially more comfortable fit in the hand(s) of a user.

The embodiment shown in FIG. 3 is particularly ergonomic, because a length of thermally conductive tubing 318 may be positioned outside and flush with the plurality of consecutive, concentric windings 312, creating a lengthwise outer ridge 326 along the at least one ergonomic, frustoconical coil 310. The lengthwise outer ridge 326 may provide indicator(s) for right thumb placement RT and/or left thumb placement LT.

A different ergonomic embodiment is shown in FIGS. 4A-4D. A coil 410 is defined by sequential windings with a lengthwise outer ridge 426, defined by a length of thermally conductive tubing positioned outside and flush with a plurality of consecutive, concentric windings, very similar to what is shown in FIG. 3. A protective covering 416 of the ergonomic, frustoconical coil(s) 410 defines an oval or teardrop shaped outer profile, which together provide a user with a shape and feel that are comfortable for hand gripping. However, the ergonomic, frustoconical coil(s) 410 are also suitable for non-handheld use.

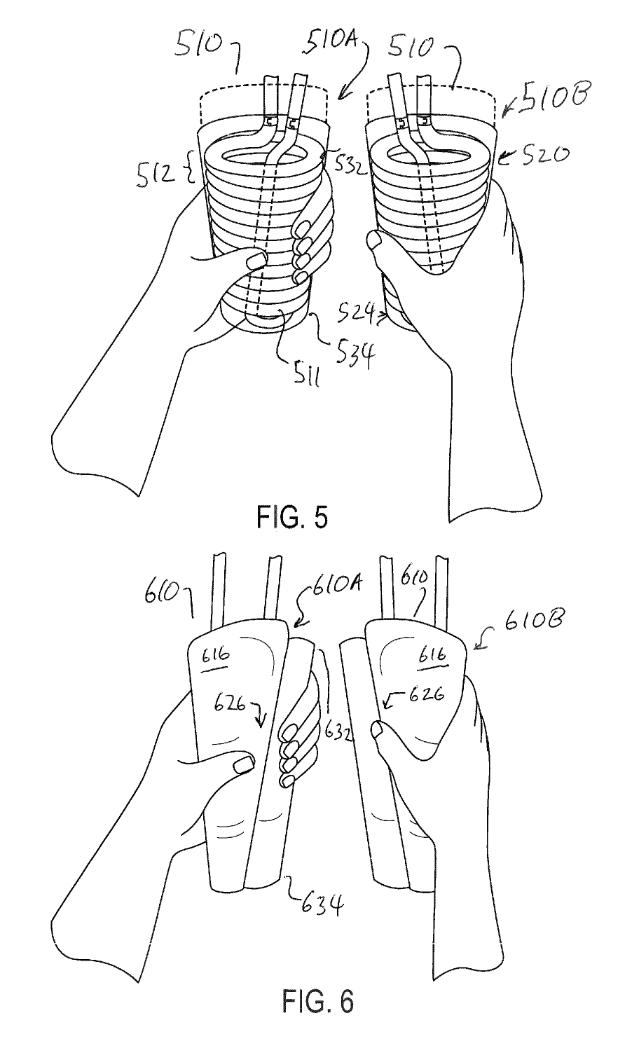

In another alternative embodiment, as shown in FIG. 5, the distal end(s) 534 of the frustoconical coil(s) 510 may be smaller than the base(s) 532 of the frustoconical coil(s). In other words, the plurality of consecutive, concentric windings 512 of thermally conductive tubing 511 may define incrementally decreasing diameters. Specifically, the first concentric winding 520 may have a first diameter of approximately 1.75-inches at the base(s) 532 of the frustoconical coil(s) 510, and the final concentric winding 524 may have a final diameter of approximately 1-inch at the distal end(s) 534 of the frustoconical coil(s) 510, or any other suitable first and final diameters. Similar to the coil(s) 10 of FIG. 1A, the size(s) of the frustoconical coil(s) 510 may vary in embodiments intended for different users, such as children and/or adults.

In the alternative embodiment shown in FIG. 6, distal end(s) 634 of the frustoconical coil(s) 610 are smaller than the base(s) 632 of the frustoconical coil(s). As discussed above, a plurality of consecutive, concentric windings of thermally conductive tubing define incrementally decreasing diameters. However, in the embodiment of FIG. 6, a lengthwise outer ridge 626 is defined by a length of thermally conductive tubing positioned outside and flush with the plurality of consecutive, concentric windings. A protective covering 616 of the ergonomic, frustoconical coil(s) 610 defines an oval or teardrop shaped outer profile, which together provide a user with a shape and feel that are comfortable for hand gripping.

The following paragraphs may provide further information regarding example embodiments.

The disclosed embodiments include coils that may be used with available hospital grade pumps to safely circulate warm or cold water. The coils are effective, safe, easy and convenient to use, inexpensive, and reusable after simply cleaning any outer smooth surfaces or by replacing a disposable sleeve.

By warming the blood in the palm of the hands through skin contact, each coil helps maintain patient temperatures in a surgical environment. The coil takes advantage of the hands' efficiency of heat exchange radiating heat better than any other body part. In exposed areas of the body such as the hands, feet and ears, blood can be shunted through arteriovenous anastomosis from the arterioles to veins bypassing capillaries and venules. Blood flow into the venous plexus can vary from just above zero to 30% of the total cardiac output.

Another important use of the coils is preparing patients for blood draw or IV start procedures which are among the most stressful events during hospitalization and laboratory testing. Proper use of the disclosed coils helps dilate the blood vessels to their maximum size thus increasing the success rate of these procedures while lessening patient distress. Patients have found that holding and squeezing the disclosed coils provides comfort and stress relief in both pre- and post-surgical areas as well as during surgery. The various embodiments of coils are is easy to use in the operating room. Simply lay a coil on the palm of a hand, or loosely wrap a coil in the hands when arms are tucked at sides or under blankets. Patients who are awake also find that holding one or more of the disclosed coils not only provides warmth but also comfort and stress relief through focused attention and squeezing motion. The coils will warm or cool and comfort a person in many situations.

The coils can also be used for heat stress events, such as heatstroke and heat exhaustion. According to OSHA standards these patients require medical care. Current treatment includes starting an IV, packing head, underarms and groin with ice and is universally disliked by patients. The disclosed coils can be used immediately, even in remote locations, without the need for specialized healthcare providers, for example to treat firefighters or victims of hypothermia or exposure. The coils can be used in remote locations with a fluid circulator, 12V battery, an AC inverter, and ice if cooling.

One method of constructing the disclosed coils is as follows. Starts by cutting approximately 100-inches of ¼-inch flexible copper tubing. Fill the cut tubing with water and freeze the water in order to avoid kinking while manipulating. One such method includes leaving an 8-inch tail at the top while then wrapping the cut tubing tightly around a wide (1.75-inch diameter) end of a 4.5-inch long tapered metal form. Continue wrapping coils tightly together down to a narrower (1-inch diameter) end of the form until the coil is approximately 5-inches in length. Slip out the form and feed the remaining top end of the copper back through the center of the coil, being careful not to kink the upper turn. Straighten and evenly trim the 2 tails at the bottom/narrow end to be about 1-inch long. The ergonomic coils are made the same way except that the copper tube is not inserted down inside the coil but instead is slightly curved down along the outside of the coil.

The outer covering is assembled using 12-inches of 3-inch flexible polyolefin tubing with a 2:1 shrink ratio. Heat shrink a 6-inch end with a small propane torch. This shrunk end fits down inside the coil, and the remaining tube folds inside out over the coil and is shrunk tight with the torch. The copper coil is now encased inside and outside allowing for easy cleaning. A variety of coil sizes are possible by simply changing the diameter and length of coil.

The plastic tubing coming from the water pump is then connected over the copper tail. A pinch hose clamp is placed over the connection securing the coil to the water pump tubing. Both handheld devices are then connected in series by 6 feet of plastic tubing and clamps. The return water line is then connected to create a continuous loop enabling water to circulate back to the pump. A “Clik Tite” connector may be inserted in the plastic pump tubing to enable easy changeover of pump accessories.

While embodiments have been particularly shown and described, many variations may be made therein. This disclosure may include one or more independent or interdependent embodiments directed to various combinations of features, functions, elements and/or properties. Other combinations and sub-combinations of features, functions, elements and/or properties may be claimed later in a related application. Such variations, whether they are directed to different combinations or directed to the same combinations, whether different, broader, narrower or equal in scope, are also regarded as included within the subject matter of the present disclosure. Accordingly, the foregoing embodiments are illustrative, and no single feature or element, or combination thereof, is essential to all possible combinations that may be claimed in this or a later application.

A method is also disclosed for regulating body temperature, comprising the following steps. Forming at least one ergonomic, frustoconical coil of thermally conductive tubing to define a plurality of consecutive, concentric windings. Bending the thermally conductive tubing from a final concentric winding to extend along the at least one ergonomic, frustoconical coil, in a direction substantially perpendicular to the concentric windings, and positioned outside and flush with the concentric windings, creating a lengthwise outer ridge along the at least one ergonomic, frustoconical coil. Connecting the at least one ergonomic, frustoconical coil to an external fluid supply by use of a connection tubing. Securing points of connection between the at least one ergonomic, frustoconical coil and the tubing, and between the connection tubing and the external fluid supply. Tightly enclosing the at least one ergonomic, frustoconical coil with a smooth, protective covering. Functionality is provided by forcing warm or cold fluid from the external fluid supply to flow through the connection tubing and the at least one ergonomic, frustoconical coil.

This method may include other steps, as well, such as completing a fluid circuit between the at least one ergonomic, frustoconical coil and the external fluid supply by use of the connection tubing, and forcing circulation of warm or cold fluid through the fluid circuit. The at least one ergonomic, frustoconical coil comprises a first coil and a second coil, the first coil and the second coil are connected in series by connection tubing and secured at points of connection, the first coil is configured to be held in a hand of a user, and the second coil is configured to be held in another hand of a user.

Another method for regulating body temperature includes the following steps. Forming at least one cylindrical coil of thermally conductive tubing. Connecting the at least one cylindrical coil to an external fluid supply by use of a connection tubing. Securing points of connection between the at least one cylindrical coil and the connection tubing, and between the connection tubing and the external fluid supply. Tightly enclosing the at least one cylindrical coil with a protective covering. The method is completed by forcing warm or cold fluid from the external fluid supply to flow through the connection tubing and the at least one cylindrical coil.

Further steps include completing a fluid circuit between the at least one cylindrical coil and the external fluid supply by use of the connection tubing, and forcing circulation of warm or cold fluid through the fluid circuit.

It is believed that the disclosure set forth herein encompasses multiple distinct inventions with independent utility. While each of these inventions has been disclosed in its preferred form, the specific embodiments thereof as disclosed and illustrated herein are not to be considered in a limiting sense as numerous variations are possible. Each example defines an embodiment disclosed in the foregoing disclosure, but any one example does not necessarily encompass all features or combinations that may be eventually claimed. Where the description recites “a” or “a first” element or the equivalent thereof, such description includes one or more such elements, neither requiring nor excluding two or more such elements. Further, ordinal indicators, such as first, second or third, for identified elements are used to distinguish between the elements, and do not indicate a required or limited number of such elements, and do not indicate a particular position or order of such elements unless otherwise specifically stated.

What is claimed is:

1. A portable heat exchanging apparatus for regulating body temperature, comprising:
at least one coil of thermally conductive tubing formed to define a plurality of consecutive, concentric windings;
a connection tubing connecting the at least one coil to an external fluid supply;
a protective covering tightly enclosing the at least one coil, providing a smooth surface for user comfort, easy sanitization, and reusability.

2. The portable heat exchanging apparatus according to claim 1, wherein the plurality of consecutive, concentric windings define incrementally increasing diameters, with a first concentric winding having a first diameter, and an adjacent concentric winding having a second diameter that is larger than the first diameter.

3. The portable heat exchanging apparatus according to claim 1, wherein a length of thermally conductive tubing bends from one of the plurality of consecutive, concentric windings to extend along the at least one coil, in a direction substantially perpendicular to an axis defined by the plurality of consecutive, concentric windings, and positioned inside the plurality of consecutive, concentric windings.

4. The portable heat exchanging apparatus according to claim 1, wherein a length of thermally conductive tubing bends from one of the plurality of consecutive, concentric windings to extend along the at least one coil, in a direction substantially perpendicular to an axis defined by the plurality of consecutive, concentric windings, and positioned outside and flush with the plurality of consecutive, concentric windings, creating a lengthwise outer ridge along the at least one coil.

5. The portable heat exchanging apparatus according to claim 1, wherein the at least one coil comprises a first coil and a second coil.

6. The portable heat exchanging apparatus according to claim 1, wherein the protective covering fully encloses the at least one coil to define a smooth contiguous surface outside of the at least one coil.

7. The portable heat exchanging apparatus according to claim 1, wherein the protective covering fully encloses the at least one coil to define a smooth contiguous surface inside of the at least one coil.

8. A portable heat exchanging apparatus for regulating body temperature, comprising:
at least one frustoconical coil of thermally conductive tubing defining a plurality of consecutive, concentric windings;
a base of the at least one frustoconical coil having a first concentric winding of thermally conductive tubing, a beginning of the thermally conductive tubing for fluid input, and an end of the thermally conductive tubing for fluid output; and
a distal end of the at least one frustoconical coil having a final concentric winding of thermally conductive tubing.

9. The portable heat exchanging apparatus according to claim 8, wherein the distal end of the at least one frustoconical coil is larger than the base of the at least one frustoconical coil.

10. The portable heat exchanging apparatus according to claim 8, wherein the plurality of consecutive, concentric windings have no substantial empty spaces there between.

11. The portable heat exchanging apparatus according to claim 8, wherein the at least one frustoconical coil comprises at least five consecutive, concentric windings.

12. The portable heat exchanging apparatus according to claim 8, wherein the at least one frustoconical coil is tightly enclosed by a protective covering, providing a smooth surface for user comfort, easy sanitization, and reusability.

13. The portable heat exchanging apparatus according to claim 8, wherein:
a connection tubing connects the at least one frustoconical coil to an external fluid supply; and
a protective covering tightly encloses the tubing.

14. The portable heat exchanging apparatus according to claim 8, wherein a length of thermally conductive tubing bends from the final concentric winding to extend along the at least one frustoconical coil, in a direction substantially perpendicular to an axis defined by the consecutive, concentric windings, and positioned inside the consecutive, concentric windings.

15. The portable heat exchanging apparatus according to claim 8, wherein a length of thermally conductive tubing bends from the final concentric winding to extend along the at least one frustoconical coil, in a direction substantially perpendicular to axis defined by the consecutive, concentric windings, and positioned outside and flush with the consecutive, concentric windings, creating a lengthwise outer ridge along the at least one frustoconical coil.

16. The portable heat exchanging apparatus according to claim 15, wherein the lengthwise outer ridge creates an ergonomic shape for the at least frustoconical one coil, suitable for hand gripping, with right or left thumb placement.

17. A method for regulating body temperature, comprising the steps of:
providing a portable heat exchanging apparatus comprising at least one coil of thermally conductive tubing;
connecting the portable heat exchanging apparatus to an external fluid supply;
forcing warm or cold fluid to flow from the external fluid supply through the at least one coil;
placing the at least one coil on an exposed body part of a user; and
using the warm or cold fluid flowing through the at least one coil to transfer heat between the portable heat exchanging apparatus and an exposed body part of a user that is in contact with the at least one coil.

18. The method for regulating body temperature according to claim 17, further comprising the step of using tubing to complete a fluid circuit between the at least one coil and the external fluid supply.

19. The method for regulating body temperature according to claim 17, further comprising the step of placing the at least one coil in a palm of a hand of a user, such that the at least one coil may be gripped in a hand of a user.

20. The method for regulating body temperature according to claim 17, wherein the at least one coil comprises a first coil and a second coil.

21. The method for regulating body temperature according to claim 17, wherein the at least one coil is configured in an ergonomic shape, suitable for hand gripping, with right or left thumb placement.
